(19) United States
(12) Patent Application Publication
(10) Pub. No.: US 2012/0221099 A1
(43) Pub. Date: Aug. 30, 2012

(54) COATED BIOLOGICAL MATERIAL HAVING IMPROVED PROPERTIES

(76) Inventors: Alexander Borck, Aurachtal (DE); Alexander Rzany, Nuernberg (DE)

(21) Appl. No.: 13/354,392

(22) Filed: Jan. 20, 2012

Related U.S. Application Data
(60) Provisional application No. 61/446,054, filed on Feb. 24, 2011.

(51) Int. Cl. A61F 2/24; B05D 5/00; C23C 14/30; C23C 14/14; B32B 9/02; B32B 9/04

(52) U.S. Cl. 623/2.1; 428/473; 428/334; 428/457; 428/446; 428/336; 427/596; 427/597; 427/2.25

(57) ABSTRACT

Some embodiments of the present invention relate to coated biological material for the manufacture of heart valve prostheses, characterized in that the surface of the biological material is covered entirely or partially with a coating which contains or is composed of a biocompatible anorganic material, and to a process for manufacturing such a coated biological material.

### CROSS REFERENCE

The present application claims priority on co-pending U.S. Provisional Application No. 61/446,054 filed on Feb. 24, 2011; which application is incorporated herein by reference.

### TECHNICAL FIELD

Some embodiments of the present invention relate to a material for the manufacture of heart valve protheses, to a process for manufacturing this material and to a valve. Other embodiments relate to coated biological materials.

### BACKGROUND

The replacement of heart valves that are no longer functional with heart valve prostheses is second only to coronary bypass surgery as the most common operation performed on the human heart.

Basically, two different types of heart valve prostheses can be used, namely mechanical or biological heart valve prostheses.

Mechanical heart valve prostheses and the accompanying drugs place considerable limitations on patients. Patients who have a mechanical heart valve must be treated with anticoagulants for the rest of their lives and are therefore continually at high risk for thromboembolic complications and bleeding.

To avoid these disadvantages and difficulties, biological heart valve prostheses were developed on the basis of human tissue (as an allograft or homograft), or animal tissue (as a xenograft). The development of biological heart valve prostheses, in particular prostheses having valve cusps composed of biological material of animal origin (“xenografts”), enables permanent anticoagulation to be eliminated. However, biological heart valves tend to calcify, and calcification can cause the prosthesis to lose functionality. It was shown that, despite exercising extensive care and caution in the preparation of the biological material, even slight traces of proteins or cells that had not been fully denatured can induce a biological response after implantation. As a result, premature calcification of the biological valve material is observed. The biological heart valve prosthesis fails and may need to be replaced. In addition to calcification, the continual mechanical load on the prostheses also poses a problem, and therefore biological heart valve prostheses usually have shorter service lives than mechanical heart valve prostheses. Many other unresolved problems in the art exist.

### SUMMARY

One of the problems to be solved by the present invention is that of reducing or avoiding one or more disadvantages of the prior art. In particular, a problem solved by some embodiments of the present invention relate to providing biological material for use in biological heart valve prostheses and other applications, which allow for long service lives of heart valve prostheses and other applications.

This and other problems are solved by embodiments of the present invention that provide coated biological material for the manufacture of heart valve prostheses and for other applications, characterized in that the surface of the biological material is covered entirely or partially by a coating that comprises or consists entirely of a biocompatible anorganic material.

### DESCRIPTION OF THE DRAWINGS

FIG. 1 shows a schematic representation of a coating system for the pulsed laser deposition (PLD) of biocompatible anorganic material onto biological material.

### DETAILED DESCRIPTION

Embodiments of the invention are directed to novel biological materials carrying a biocompatible anorganic material coating, as well as processes for making the same. Some embodiments find particular utility when used as components of heart valves or processes for making the same. Many other applications will be apparent to those knowledgeable in the art, however. For purposes of illustrating particular features of invention embodiments, discussion of heart valves will be made. It will be appreciated, however, that the present invention is not limited to heart valves or processes for making the same.

By providing a coating of a biocompatible anorganic material, the valve material made of the coated biological material according to an invention embodiment is much more insensitive to calcification. In addition, rejection reactions after implantation of heart valve prostheses made thereof are markedly reduced. Any immunogenic components of the biological material that may be present are protected (and isolated from) by the biocompatible anorganic coating against direct or other contact with the surrounding environment, which may be the immune system of the recipient or patient. Moreover, the coating of a biocompatible anorganic material helps to mechanically stabilize the biological material and valve material composed thereof against wear, thereby making it possible to extend service lives of biological heart valve prostheses which comprise or are composed of the coated biological material according to the invention. As a result, younger patient populations can be provided with heart valve prostheses containing the coated biological material according to the invention, and aftercare is not required until much later or possibly not at all. These are just some of the important benefits and advantages achieved through invention embodiments.

For the purposes of the present invention, the expression “biological material” refers to biological material that can be used to manufacture heart valve prostheses, in particular to manufacture biological heart valve prostheses. This is preferably biological material that is used to manufacture valve cusps of heart valve prostheses. In that particular case, the biological material is of biological origin, and has therefore been obtained from tissue or components of human or animal organisms or organs, or was formed in vitro using human or animal cells, using “tissue engineering” techniques, for example. The biological material is preferably obtained from pericardial, cardiac, venous and/or aortic tissue, and particularly preferably the biological material is obtained from pericardial tissue, heart valve tissue, venous and/or aortic valve tissue. Since it is often not possible to provide a sufficient quantity of biological material of human origin, biological material of xenogenic origin is used in particular. In that case, the biological material is preferably of porcine, bovine, or equine origin. For example, the biological material can be obtained from heart valve or pericardial material from swine, or from pericardial tissue from cattle or horses. It is likewise possible to obtain the biological tissue from venous or aortic tissue, wherein it is possible to use possibly human allogenic or even autogenic tissue.

The biological material may have been treated to ensure the desired preservation and mechanical stability, and to minimize the risk of calcifications and rejection reactions. For this reason, fixation, preservation, and/or decellularization is often carried out. Suitable techniques are known to a person skilled in the art. In the pretreatment of the biological material, protein components in particular of the extracellular matrix are cross-linked/affixed, cells are killed/destroyed, and immunogenic, cellular residual components are removed, and so a biological material is obtained that is composed substantially of affixed, decellularized, extracellular material. Preferably the biological material comprises or consists entirely of extracellular material, particularly preferably of decellularized extracellular matrix. This extracellular material preferably contains mainly collagen and elastin components.

The coated biological material according to the invention is characterized in that the surface of the biological material is covered entirely or partially with a coating which comprises or consists entirely of a biocompatible anorganic material.

According to the invention, a coating refers to the application, at least in sections, of the components of the coating onto the surface of the biological material. Preferably the entirety of the portion of the surface of the biological material is covered with the coating which comes into contact with components of the immune system or with flowing blood of the receiving organism immediately after implantation of the heart valve prosthesis. A layer thickness may be as desired, with some examples including in the range of 10 nm to 100 μm, 50 nm to 15 μm, 100 nm to 3 μm, or other thicknesses, including less than 10 nm and greater than 100 μm. It is thereby ensured that the coating is thick enough to permanently rule out direct contact between the surface coated therewith and the external environment, which in many applications are components of the recipient organism after implantation. The layer thickness is also selected such that the mechanical properties of the biological material are not substantially impaired in regard to flexibility and toughness, and in regard to the suitability thereof for use as valve material. Although thicknesses will vary with application, a good balance and achievement of these desired attributes has been discovered to exist in many applications by setting the thickness between about 100 nm to 3 μm. The coating can be applied directly and immediately onto the surface of the biological material. The coating can be applied in a number of different steps, with an example including using a pulsed laser deposition (PLD) process. The coating can be created as a single-layered system or a multiple-layered system, it being possible for different layers to be composed of different materials. In some embodiments of the invention, then, a first biocompatible anorganic coating layer may be applied having a first composition through PLD, with a second (and further) biocompatible anorganic coating layers subsequently applied. Description herein of any coating layer and its properties (including, for example, composition, layer thickness, and other) will be appreciated to also apply to other layers in a multiple layer application.

The coating comprises or consists entirely of a biocompatible anorganic material. A biocompatible anorganic material is a nonliving material that is used for a medical application and interacts with biological systems. A prerequisite for the use of a material that comes in contact with the body environment when used as intended is its biocompatibility. “Biocompatibility” refers to the capability of a material to evoke an appropriate tissue response in a specific application. This includes an adaptation of the chemical, physical, biological, and morphological surface properties of an implant to the recipient tissue, with the objective of achieving a clinically desired interaction. Preferably, biocompatible anorganic materials that are substantially bioinert are used for the coating. “Bioinert materials” are those materials that remain substantially intact and exhibit no significant biocorrosion after implantation, for the planned service life of the heart valve prosthesis. Artificial plasma, as prescribed according to EN ISO 10993-15:2000 for biocorrosion assays (composition 6.8 g/l NaCl , 0.2 g/l CaCl₂, 0.4 g/l KCl, 0.1 g/l MgSO₄, 2.2 g/l NaHCO₃, 0.126 g/l Na₂HPO₄, 0.026 g/l NaH₂PO₄), is used as a testing medium to investigate the corrosion behavior of a material under consideration. A sample of the material to be investigated is stored in a closed sample container with a defined quantity of the testing medium at 37° C. The samples are removed and examined in a known manner for traces of corrosion at time intervals defined according to the anticipated corrosion behavior, of a few days up to multiple months or years. The artificial plasma according to EN ISO 10993-15:2000 corresponds to a medium similar to blood and thus represents a way to reproducibly simulate a physiological environment within the scope of the invention. A material is considered to be bioinert in particular when the material has corroded by less than 10% in the aforementioned test after a period of 12 months.

The biocompatible anorganic material can be comprised or consist entirely of a metal, a metal alloy, a polymer, a ceramic material, or amorphous carbon, or can include such a material. In the present context, an alloy refers to a metallic microstructure having a metal as the main component, in particular iron, tantalum, titanium, nickel, cobalt, chromium, or molybdenum. The main component is the alloy component that comprises the largest percentage by weight of the alloy. In some alloys, a portion of the main component is more than 50% by weight, and in others more than 70% by weight.

Biocompatible metals and metal alloys for the coating can contain stainless steels (316L, for example), Co/Cr alloys (such as CoCrMo casting alloys, CoCrMo forging alloys, CoCrWNi forging alloys, and CoCrNiMo forging alloys), pure titanium and titanium alloys (e.g. CP titanium, TiAl6V4 or TiAl6Nb7), nickel or nickel alloys, tantalum or tantalum alloys, gold alloys and/or amorphous carbon, “diamond-like carbon” or DLC. In some embodiments the biocompatible anorganic material is selected from tantalum, nickel, titanium, Nitinol, stainless steel, alloys thereof, Co/Cr alloys and/or amorphous carbon having an sp² hybridization portion of 30% to 70%, or an sp³ hybridization portion of 70% to 30% (“diamond-like carbon”, or DLC); and the hydrogen content of these layers is between 10% and 65%. Such amorphous carbon layers (DLC) have excellent mechanical and chemical properties and have high biocompatibility. Due to the combination of great hardness, low friction coefficient, and chemical resistance, it has been discovered that this class of layers is well suited for use in invention embodiments. The friction coefficient is lower than that of typical component materials, such as steel alloys or layers of ceramic, mechanically resistant material, by a factor of 3 to 10, for example. It has been discovered that the combination of great hardness and the lowest friction coefficients is well suited for biomedical implants of the invention such as heart valve prostheses. The result is reduced or at least markedly reduced wear (abrasive wear) and corrosion (adhesive wear).

Embodiments of the present invention also relate to valve cusps for use in a heart valve prosthesis, the valve cusp being composed of or containing coated biological material according to the invention.

A further subject matter of the present invention is a heart valve prosthesis which is characterized in that it contains or is composed of this coated biological material according to the invention, and/or comprises valve cusps according to the invention.

Embodiments of the present invention also relate to a process for the manufacture of coated biological material according to the invention. The process according to the invention is characterized in particular in that the biological material is provided with a coating that comprises or consists entirely of a biocompatible anorganic material. This is achieved in many embodiments by covering the surface of the biological material, entirely or partially, with a coating containing or composed of a biocompatible anorganic material using pulsed laser deposition (PLD).

Pulsed laser deposition or laser ablation (PLD) is a technical process for use to apply metallic layers onto polymers in a gentle manner. Pulsed laser deposition is a special case of physical vapor deposition (PVD). In pulsed laser deposition, material to be transferred from a target is converted to the gas phase via ablation, is focused in the form of a plasma plume, is radiated onto the substrate to be coated, and is deposited there. The process is characterized by the high energy density of the laser pulse, which results in excitation and ablation of the material to be transferred in a very small volume. The process of ablation is a non-equilibrium process which is not dependent on the vapor pressures of the individual components, in contrast to the classical thermal evaporation of materials. Instead, the atoms of the material excited by the laser pulse immediately transition into the gas phase without energy being transferred to adjacent atoms. As a consequence thereof, the material to be transferred does not melt, and the deposition rate is independent of the chemical nature of the individual material components. In addition, the ablated material can be deposited onto biological material as the substrate, due to the low thermal energy. A unique feature that distinguishes pulsed laser deposition from many other coating techniques is deposition in thermodynamic non-equilibrium. This means that, unlike thermal evaporation, for example, the material to be transferred is not released in accordance with the evaporation enthalpy thereof. The effectiveness of the coating is independent of the specific substance. Using pulsed laser deposition it is therefore also possible to deposit alloys of the type present in the target onto the substrate without the stoichiometry being changed. Due to the focused laser beam, the absence of thermal load, and the pulsed deposition, it is also possible to use small targets and even coatings as the source (target) for the material to be transferred, which reduces the amount of material used in the case of expensive samples in particular. In this manner, it has been discovered that use of PVD is a highly effective method of applying coatings of the invention and leads to particular advantages and benefits (in at least many applications) that are not achieved when using other application methods (although other methods will be suitable for some other applications and embodiments).

The development of the UV laser has resulted in high laser energies of up to 1 J/pulse, and some lasers achieve pulse rates of up to 300 Hz. Using pulsed laser deposition, deposition rates of at least 0.01 nm/pulse can be generated for metals. It is therefore possible to achieve rates of 300 nm/minute or layer thicknesses of 1.5 μm in 5 minutes, thereby resulting in useful coating times overall.

In one example process for pulsed laser deposition according to the invention, a pulsed laser is used at:
a frequency of 1 Hz to 300 Hz, particularly preferably at a frequency of 10 Hz to 100 Hz; and/or
a pulse duration of 0.1 ns to 200 ns (ns=nanosecond), particularly preferably 1 ns to 100 ns; and/or
an energy density of 0.01 J/cm² to 100 J/cm², particularly preferably 0.1 J/cm² to 30 J/cm², in particular 1 J/cm² to 15 J/cm²

These particular values have been discovered to lead to highly beneficial results in at least some applications. It is basically possible to use any type of laser in that case; for pulsed laser deposition in particular, it is possible to use pulsed excimer lasers, CO₂ lasers or Nd:YAG lasers.

In the process according to at least some embodiments of the invention, the pulsed laser deposition takes place in a vacuum. A vacuum is understood to be a state in which a gas pressure of less than 300 mbar is present. In some embodiments, pulsed laser deposition preferably takes place at a pressure of no more than 1 mbar, in others at a pressure of 0.1 mbar to 10⁻¹⁰ mbar, and in other embodiments other pressures.

In the process according to some invention embodiments, pulsed laser deposition is carried out under aseptic conditions. This ensures that the requirements on the sterility of the coated biological material that is created can be guaranteed.

In some embodiments of the process according to the invention, the biocompatible anorganic material is deposited onto the biological material up to a mean layer thickness of 1 nm to 100 μm, in others 10 nm to 15 μm, in others 100 nm to 3 μm, and in others other thicknesses including less than 1 nm or greater than 100 μm.

In the process according to the invention, the biocompatible anorganic material to be transferred can be comprised of or consist entirely of a metal, a metal alloy, a polymer, a ceramic material or amorphous carbon, preferably tantalum, nickel, titanium, Nitinol, stainless steel, alloys thereof, Co/Cr alloys and/or amorphous carbon having an sp² hybridization portion of 30% to 70%, or an sp³ hybridization portion of 70% to 30% (“diamond-like carbon” or DLC).

By changing the coating source and, therefore, the material to be deposited, for example by using a rotational holder having a plurality of coating sources in the recipient, it is possible to deposit a multiple-layered system onto the biological material, thereby providing another way to optimize the physical/chemical properties of the surface of the coated biological material according to the invention. In these embodiments, each of the multiple deposited layers have a different composition and are deposited independently of one another.

Preferably, the biological material is coated using the process described above before it is used in the manufacture of valve cusps or in the assembly of heart valve prostheses.

The process according to the invention makes it possible to apply a biocompatible anorganic layer directly onto the biological material which imparts the desired mechanical properties of heart valves. Due to the anorganic layer, the biological signature of the possibly xenogenic, biological starting material is blocked or destroyed, thereby preventing rejection reactions. In addition, calcification of the coated biological material is prevented by way of the anorganic surface thereof. The gentle coating of biological material with biocompatible anorganic materials such as metals or DLC layers makes it possible to trigger the body's immune response and solve the problem of calcification while retaining the mechanical properties of the biological material. The mechanical properties of the biological material are retained while optimizing the surface in regard to wear and biologically/chemically inert behavior. Biological signatures of proteins and cells are masked by the anorganic coating and removed from the immune response of the recipient's body. Since the approach described above does not have any of the limitations in regard to mechanical properties and material thickness that are relevant to the clinical application, a significant improvement of biological heart valves on the basis of biological material is achieved.

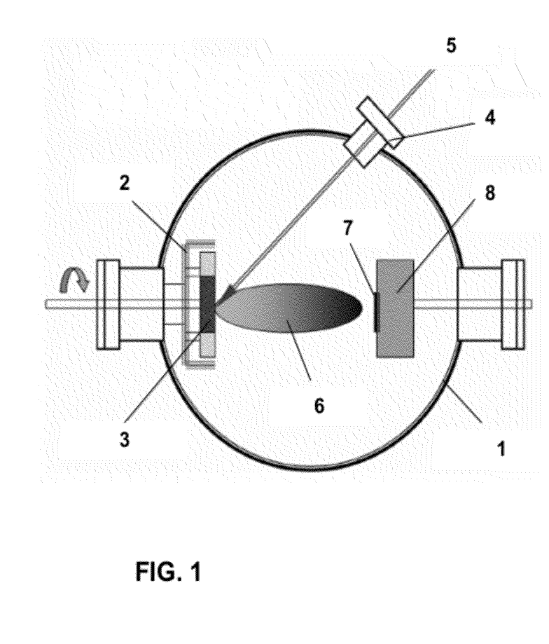

Some features of some embodiments of the invention are explained in greater detail below with reference to FIG. 1.

The basic design of one example coating system for the pulsed laser deposition (PLD) of biocompatible anorganic material onto biological material is depicted schematically in FIG. 1. Target 3 is fastened on a rotating carousel 2 in a vacuum chamber 1. Target 3 comprises or consists essentially of the material to be transferred. Target 3 is shot with a laser beam 5 through a window 4 in vacuum chamber 1. Material to be transferred from target 3 is converted by laser beam 5 to the gas phase by ablation and, focused in the form of a plasma plume 6, is radiated and deposited onto substrate 7 to be coated, i.e. the biological material to be coated, which is mounted on a holder 8.

### Embodiment 1

### Pericardium with Titanium Coating

One embodiment is the coating of prepared pericardial tissue from swine with a metallic layer of biocompatible titanium. For deposition, a PLD system is used at room temperature with a 50 Hz Nd:YAG laser (532 nm wavelength) at an energy density of approximately 5 J/cm². A sheet of pure titanium (99.99% purity) is used as the coating source. The residual pressure in the vacuum chamber is 10⁻⁶ mbar. The distance between the coating source and the porcine pericardium to be coated is 3 cm. The deposition rate is 0.025 nm/coating pulse, or 75 nm/minute, under these conditions. Layer thicknesses of more than one micrometer can be attained within 15 minutes.

Using the same coating system and identical deposition parameters, practically any other metal can be deposited onto pericardial material using pulsed laser deposition.

### Embodiment 2

### Pericardium with DLC Coating

A second embodiment is coating using biocompatible, diamond-like carbon (DLC). A two-staged process is necessary for some DLC layers. In the first step, a hydrocarbon layer is applied using a plasma process. As the process continues, the hydrogen content in the plasma polymer is reduced until the carbon is present in the diamond configuration. The process temperatures are greatly exceed 100° C. and are much too high for coating biological pericardial material. Using pulsed laser deposition, carbon and hard carbon coatings can be attained in the range of room temperature.

For deposition, a PLD system is used at room temperature with a 20 Hz Nd:YAG laser (1064 nm wavelength) with a pulse duration of 25 ns at an energy density of approximately 10 J/cm². A bar of pure graphite (99.99% purity) is used as the coating source, and is moved during coating at a rate of 2 mm/min to minimize the formation of craters caused by material removal. The residual pressure in the coating chamber is 10⁻⁶ mbar. The distance between the coating source and the porcine pericardium to be coated is 5 cm. The deposition rate is 0.03 nm/coating pulse, or 18 nm/minute, under these conditions. Layer thicknesses of more than 500 nm can be attained within 15 minutes.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A coated biological material for the manufacture of heart valve prostheses, characterized in that the surface of the biological material is covered entirely or partially with a coating comprising a biocompatible anorganic material.

2. The coated biological material according to claim 1, wherein the biological material comprises an extracellular matrix.

3. The coated biological material according to claim 1, wherein the biological material is obtained from one or more of pericardial, cardiac, venous and aortic tissue.

4. The coated biological material according to claim 1, wherein the biological material is one of human, porcine, bovine, or equine origin.

5. The coated biological material according to claim 1, wherein the coating of a biocompatible anorganic material has a mean layer thickness of 10 nm to 100 μm.

6. The coated biological material according to claim 1, wherein the biocompatible anorganic material comprises one or more of a metal, a metal alloy, a polymer, a ceramic material or amorphous carbon, preferably tantalum, nickel, titanium, Nitinol, stainless steel, alloys thereof, Co/Cr alloys and amorphous carbon having an sp² hybridization portion of 30% to 70%, and amorphous carbon having an sp³ hybridization portion of 70% to 30% (“diamond-like carbon” or DLC).

7. The biological material of claim 1 wherein the material is configured as a valve cusp for use in a heart valve prosthesis.

8. The biological material of claim 1 wherein the material is configured as a valve cusp of a heart valve prosthesis.

9. A process for manufacturing a coated biological material comprising the steps of using pulsed laser deposition (PLD) to deposit a coating on at least a portion of the biological material, the coating comprising a biocompatible anorganic material.

10. The process according to claim 9, wherein a pulsed laser having a frequency of 1 Hz to 300 Hz, a pulse duration of 0.1 ns to 200 ns and/or an energy density of 0.1 J/cm² to 30 J/cm² is used for the pulsed laser deposition.

11. The process according to claim 9, wherein one of a pulsed excimer laser, a CO₂ laser and an Nd:YAG laser is used for the pulsed laser deposition.

12. The process according to claim 9, wherein the pulsed laser deposition takes place in a vacuum at a pressure that does not exceed 1 mbar.

13. The process according to claim 9, wherein the pulsed laser deposition takes place under aseptic conditions.

14. The process according to claim 9, wherein the biocompatible anorganic material is deposited onto the biocompatible anorganic material with a mean layer thickness of 10 nm to 100 μm.

15. The process according to claim 9, wherein the biocompatible anorganic material is comprises one of a metal, a metal alloy, a polymer, a ceramic material or amorphous carbon, preferably tantalum, nickel, titanium, Nitinol, stainless steel, alloys thereof, Co/Cr alloys and amorphous carbon having an sp² hybridization portion of 30% to 70%, or an sp³ hybridization portion of 70% to 30% (“diamond-like carbon” or DLC).

16. The process according to claim 9 wherein the layer consists entirely of the biocompatible anorganic material and wherein the biocompatible anorganic material comprises amorphous carbon having one of an sp² hybridization portion of 30% to 70% and an sp³ hybridization portion of 70% to 30%.

17. The process according to claim 9 wherein the layer consists entirely of the biocompatible anorganic material and wherein the mean layer thickness is 100 nm to 3 μm.

18. The coated biological material according to claim 1, wherein:
the biological material is xenogenic;
the layer consists entirely of the biocompatible anorganic material;
the biological material comprises decellularized extracellular matrix; and,
the layer blocks the biological signature of the xenogenic biological material, thereby preventing rejection reactions when the biological material is in a physiologic environment.

19. The coated biological material according to claim 1, wherein:
the coating consists entirely of the biocompatible anorganic material;
the layer has a mean layer thickness of 100 nm to 3 μm; and,
wherein any imunogenic components of the biological material are thereby insulated from exposure to the surrounding environment by the biocompatible anorganic coating.

20. A process for manufacturing a valve cusp of a heart valve prosthesis comprising the steps of:
providing a valve cusp made of a biologic material comprising a decellulariced extracellular matrix;
operating pulsed laser deposition (PLD) at a frequency of 1 Hz to 300 Hz, a pulse duration of 0.1 ns to 200 ns and an energy density of 0.1 J/cm² to 30 J/cm² to coat the surface of the valve cusp with a coating layer having a thickness of between about 100 nm to 3 μm, the layer consisting entirely of a biocompatible anorganic material that comprises amorphous carbon having one of an sp² hybridization portion of 30% to 70% and an sp³ hybridization portion of 70% to 30%; and,
wherein any imunogenic components of the valve cusp are thereby insulated from exposure to the surrounding environment by the biocompatible anorganic coating.
